(19) United States
(12) Patent Application Publication
(10) Pub. No.: US 2017/0143302 A1
(43) Pub. Date: May 25, 2017

(54) ULTRASONIC PROBE AND ULTRASONIC IMAGE APPARATUS

(71) Applicant: Seiko Epson Corporation, Tokyo (JP)

(72) Inventors: Daisuke NAKANISHI, Matsumoto (JP); Robina ATSUCHI, Fujimi (JP); Hiroshi MATSUDA, Gifu (JP)

(21) Appl. No.: 15/348,092

(22) Filed: Nov. 10, 2016

(30) Foreign Application Priority Data
Nov. 25, 2015 (JP) 2015-229388

(51) Int. Cl. A61B 8/00; A61B 8/08; A61B 8/14

(52) U.S. Cl. CPC A61B 8/4209; A61B 8/14; A61B 8/4444; A61B 8/461; A61B 8/5207

(57) ABSTRACT

An ultrasonic probe includes an ultrasonic wave detection portion, a fixation base to which the ultrasonic wave detection portion is fixed, a casing from which the ultrasonic wave detection portion is exposed and into which the fixation base is built, and an elastic first connection portion that connects the fixation base to the casing, in which the first connection portion includes a first arm extending in a first direction and a second arm extending in a second direction intersecting the first direction, in which one end of the first connection portion is in contact with the fixation base, and the other end of the first connection portion is in contact with the casing, and in which the first direction and the second direction are the same as directions in which a surface of the fixation base and a surface of the casing facing each other extend.

### BACKGROUND

1. Technical Field

The present invention relates to an ultrasonic probe and an ultrasonic image apparatus.

2. Related Art

An ultrasonic image apparatus causes ultrasonic waves to be incident on a subject such as a structural body or a human body. The ultrasonic image apparatus detects reflected waves from a tissue or a portion with different hardness of the subject, and displays a tomographic image of the subject on the basis of the reflected waves. A phase of a reflected wave changes depending on a depth or a size of a tissue in the subject. It is possible to examine a state of a tissue or an injury inside the subject by observing a signal waveform of the reflected wave. Transmission of ultrasonic waves is performed by applying a high voltage pulse to a piezoelectric element provided in an ultrasonic probe which is brought into contact with a subject. A frequency of the ultrasonic wave is about several hundreds of KHz to several tens of MHz, and is set by an examiner selecting an ultrasonic probe to be applied according to a size of a subject or a required examination resolution. Vibration caused by a reflected wave is converted into a voltage by the piezoelectric element of the ultrasonic probe, and the voltage of the piezoelectric element is amplified by an amplification circuit connected to the piezoelectric element.

A silicon substrate provided with a plurality of piezoelectric elements is used for the ultrasonic probe. The piezoelectric elements are formed on the substrate by using a photolithographic method. The silicon substrate is a fragile material, and is cracked when being applied with impact. As a countermeasure therefor, there is a need of a structure of attenuating impact applied to a casing when the ultrasonic probe falls down.

The ultrasonic probe is used to examine internal organs of a human or an animal. The internal organs are protected by bones such as ribs in the human or the animal. Therefore, in order to examine an internal organ, the ultrasonic probe may be pushed between a bone and the internal organ. In this case, if the ultrasonic probe is large, the ultrasonic probe cannot be pushed between the bone and the internal organ, and thus the ultrasonic probe cannot be disposed at a location which is desired to be examined. Therefore, a small ultrasonic probe has good operability, and thus the ultrasonic probe can be disposed at a location which is desired to be examined.

JP-A-2014-146883 discloses an ultrasonic image apparatus provided with an ultrasonic probe. In JP-A-2014-146883, the ultrasonic probe includes an ultrasonic element array substrate on which ultrasonic elements transmitting and receiving ultrasonic waves are provided. The ultrasonic element array substrate is formed by using a silicon substrate, and is about 150 μm to 200 μm thick. Thus, the ultrasonic element array substrate is a component which is easily cracked. The ultrasonic element array substrate is provided on a support member. The support member is fixed to an exterior head section.

In the ultrasonic probe disclosed in JP-A-2014-146883, the support member is fixed to the exterior with screws. Thus, when the ultrasonic probe falls down, and therefore impact is applied to a casing, the impact is easily forwarded to the ultrasonic element array substrate.

A size of the head section of the ultrasonic probe is a size obtained by adding a thickness of the casing to a size of the ultrasonic element array substrate and a size of a screw-fixed region. In this case, the ultrasonic probe has a size to be hardly operated. Therefore, it is desirable to provide a small-sized ultrasonic probe which has good operability by reducing impact applied to an ultrasonic element array substrate even when the impact is applied to a casing.

### SUMMARY

An advantage of some aspects of the invention is to solve at least a part of the problems described above, and the invention can be implemented as the following forms or application examples.

### Application Example 1

An ultrasonic probe according to this application example includes an ultrasonic wave detection portion that includes an ultrasonic element performing at least one of transmission and reception of an ultrasonic wave; a fixation base to which the ultrasonic wave detection portion is fixed; a casing from which the ultrasonic wave detection portion is exposed and into which the fixation base is built; and a first connection portion that connects the fixation base to the casing, and is elastic, in which the first connection portion includes a first arm extending in a first direction and a second arm extending in a second direction intersecting the first direction, in which one end of the first connection portion is in contact with the fixation base, and the other end of the first connection portion is in contact with the casing, and in which the first direction and the second direction are the same as directions in which a surface of the fixation base and a surface of the casing facing each other extend.

According to this application example, the ultrasonic probe includes the ultrasonic wave detection portion, the fixation base, the casing, and the first connection portion. The ultrasonic wave detection portion includes an ultrasonic element performing at least one of transmission and reception of an ultrasonic wave. The ultrasonic wave detection portion is fixed to the fixation base. The ultrasonic wave detection portion is exposed from the casing, and the fixation base is built into the casing. The first connection portion is elastic and connects the fixation base to the casing.

The first connection portion includes the first arm extending in the first direction and the second arm extending in the second direction intersecting the first direction. The first arm and the second arm are elastic and thus function as springs. The first arm and the second arm intersect each other, and thus has elasticity with respect to force applied in any three-dimensional force. One end of the first connection portion is in contact with the fixation base, and the other end of the first connection portion is in contact with the casing. Therefore, the first connection portion is deformed when impact is applied to the casing, and can thus reduce impact applied to the ultrasonic wave detection portion from the casing.

The first arm and the second arm are located between the fixation base and the casing, and thus the first direction and the second direction are the same as directions in which a surface of the fixation base and a surface of the casing facing each other extend. Therefore, since a gap between the fixation base and the casing can be reduced, a size of the ultrasonic probe can be reduced.

### Application Example 2

In the ultrasonic probe according to the application example, the fixation base may be long in a third direction, the first connection portion may be connected to the casing on both sides of the fixation base in the third direction, and the ultrasonic probe may further include a second connection portion that connects the fixation base to the casing in a fourth direction intersecting the third direction, and that is elastic.

According to this application example, the fixation base is long in a third direction. The first connection portion is connected to the casing on both sides of the fixation base in the third direction. Consequently, the first connection portion can favorably reduce impact in the third direction. The ultrasonic probe includes the second connection portion, and the second connection portion connects the fixation base to the casing in the fourth direction intersecting the third direction, and is elastic. Consequently, the second connection portion can favorably reduce impact in the fourth direction. As a result, the ultrasonic probe can reduce impact in two directions including the third direction and the fourth direction.

### Application Example 3

The ultrasonic probe according to the application example may further include a vibration control member that is provided between the fixation base and the casing so as to attenuate vibration.

According to this application example, the vibration control member is provided between the fixation base and the casing, and the vibration control member attenuates vibration. Therefore, the ultrasonic probe can reduce influence of vibration caused by impact when the impact is applied thereto.

### Application Example 4

In the ultrasonic probe according to the application example, the first connection portion may be provided at three or more locations.

According to this application example, the first connection portion is provided at three or more locations. Since the fixation base is fixed to the casing at three or more locations via the first connection portion, it is possible to reduce that the fixation base is swung due to impact.

### Application Example 5

In the ultrasonic probe according to the application example, the ultrasonic wave detection portion may be exposed from the casing, and force applied when the ultrasonic wave detection portion is pressed may be received by the first connection portion.

According to this application example, the ultrasonic wave detection portion is exposed from the casing. The ultrasonic wave detection portion is pressed onto the subject. Force applied when the ultrasonic wave detection portion is pressed is received by the first connection portion. Therefore, it is possible to prevent the ultrasonic wave detection portion from being moved to the inside of the casing when the ultrasonic wave detection portion is pressed onto the subject.

### Application Example 6

In the ultrasonic probe according to the application example, the first connection portion may be conductive, and may relay a ground wiring of the ultrasonic wave detection portion.

According to this application example, the first connection portion is conductive. The first connection portion relays the ground wiring of the ultrasonic wave detection portion. Therefore, even when electrical noise is applied to the ultrasonic wave detection portion, the electrical noise is made to pass through the first connection portion so as to be removed.

### Application Example 7

An ultrasonic image apparatus according to this application example includes an ultrasonic probe; an image data calculation unit that calculates tomographic image data of a subject by using a reflected wave signal output from the ultrasonic probe; and an image display unit that displays a tomographic image of the subject on the basis of a result calculated by the image data calculation unit, in which the ultrasonic probe is the ultrasonic probe according to any one of the application examples.

According to this application example, the ultrasonic image apparatus includes the ultrasonic probe, the image data calculation unit, and the image display unit. The ultrasonic probe transmits an ultrasonic wave to a subject. The ultrasonic probe receives a reflected wave of the ultrasonic wave which is reflected inside the subject, and outputs a reflected wave signal to the image data calculation unit. The image data calculation unit calculates tomographic image data of the subject by using the reflected wave signal, and outputs a tomographic image to the image display unit. The image display unit displays the tomographic image of the subject.

As the ultrasonic probe, the above-described ultrasonic probe is used. The above-described ultrasonic probe has impact resistance, a small size, and good operability. Therefore, the ultrasonic image apparatus can be provided as an apparatus including the ultrasonic probe having impact resistance, a small size, and good operability.

### BRIEF DESCRIPTION OF THE DRAWINGS

The invention will be described with reference to the accompanying drawings, wherein like numbers reference like elements.

FIG. 1 is a schematic side sectional view illustrating a structure of an ultrasonic probe according to a first embodiment.

FIG. 2 is a schematic bottom view illustrating a structure of the ultrasonic probe.

FIG. 3 is a schematic plan sectional view illustrating a structure of a head section.

FIG. 4 is a main portion schematic diagram illustrating a structure of a first connection portion.

FIG. 5 is a main portion schematic diagram illustrating a structure of the first connection portion.

FIG. 6 is a main portion schematic diagram for explaining a structure of a second fixation portion of a second beam.

FIG. 7 is a main portion schematic diagram for explaining a structure of a third beam.

FIG. 8 is a main portion schematic diagram for explaining a structure of a first fixation portion of a first beam.

FIG. 9 is a schematic diagram for explaining an operation of the first connection portion.

FIG. 10 is a schematic diagram for explaining an operation of the first connection portion.

FIG. 11 is a schematic diagram for explaining an operation of the first connection portion.

FIG. 12 is a schematic diagram for explaining an operation of the first connection portion.

FIG. 13 is a schematic diagram for explaining an operation of the first connection portion.

FIG. 14 is a schematic diagram for explaining an operation of the first connection portion.

FIG. 15 is a schematic diagram for explaining an operation of a second connection portion.

FIG. 16 is a schematic diagram for explaining an operation of the second connection portion.

FIG. 17 is a schematic perspective view illustrating a structure of an ultrasonic image apparatus according to a second embodiment.

FIG. 18 is a schematic plan sectional view for explaining arrangement of a first connection portion according to a modification example.

FIG. 19 is a schematic side sectional view for explaining a structure of an ultrasonic probe according to a modification example.

### DESCRIPTION OF EXEMPLARY EMBODIMENTS

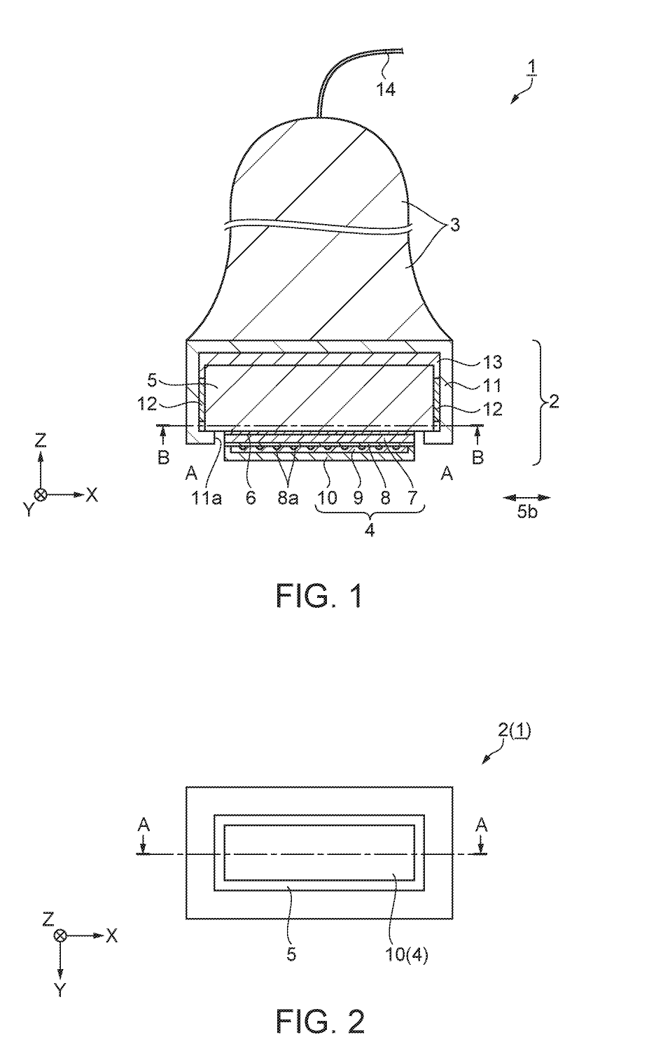

In the present embodiment, characteristic examples of an ultrasonic probe will be described with reference to FIGS. 1 to 19. Respective members in the drawings are illustrated in different scales so as to have recognizable sizes in the drawings.

### First Embodiment

An ultrasonic probe according to the present embodiment will be described with reference to FIGS. 1 to 16. FIG. 1 is a schematic side sectional view illustrating a structure of an ultrasonic probe, and FIG. 2 is a schematic bottom view illustrating a structure of the ultrasonic probe. FIG. 1 is a view taken along the line A-A in FIG. 2. As illustrated in FIGS. 1 and 2, an ultrasonic probe 1 includes a head section 2 and a holding section 3. An operator holds the holding section 3 and operates the ultrasonic probe 1.

The head section 2 includes an ultrasonic wave detection portion 4, and a fixation base 5 for fixing the ultrasonic wave detection portion 4. The ultrasonic wave detection portion 4 and the fixation base 5 are adhered to each other via a sticky tape 6. The ultrasonic wave detection portion 4 has a rectangular plate shape, and a thickness direction of the ultrasonic wave detection portion 4 is set as a Z direction. A longitudinal direction of the ultrasonic wave detection portion 4 is set as an X direction, and a direction perpendicular to the X direction in a planar direction of the ultrasonic wave detection portion 4 is set as a Y direction. The fixation base 5 also has a long shape in the X direction in the same manner as the ultrasonic wave detection portion 4. A longitudinal direction of the fixation base 5 is assumed to be a third direction 5b. The third direction 5b is a direction which is the same as the X direction.

The ultrasonic wave detection portion 4 has a structure in which a back plate 7, an ultrasonic element array substrate 8, an acoustic matching layer 9, and an acoustic lens 10 overlap each other in this order. The ultrasonic element array substrate 8 is a substrate obtained by disposing ultrasonic elements 8a on a silicon substrate in an array form. Arrangement of the ultrasonic elements 8a is not particularly limited, but, in the present embodiment, the ultrasonic elements 8a are disposed in a matrix of eight rows in the Y direction and twelve columns in the X direction. The ultrasonic elements 8a are elements which perform at least one of transmission and reception of ultrasonic waves. As the ultrasonic elements 8a, a single element may perform transmission and reception of ultrasonic waves. The ultrasonic elements 8a may be formed of elements which perform only transmission of ultrasonic waves, and elements which perform only reception of ultrasonic waves. In addition, the ultrasonic elements 8a may be formed of elements which perform only transmission of ultrasonic waves, elements which perform only reception of ultrasonic waves, and elements which perform transmission and reception of ultrasonic waves. A thickness of the ultrasonic element array substrate 8 is about 150 μm to 200 μm.

The back plate 7 suppresses residual vibration of the ultrasonic element array substrate 8. As the back plate 7, a silicon substrate having a thickness of about 500 μm to 600 μm is used. As the back plate 7, a metal plate may be used in addition to the silicon substrate.

The acoustic matching layer 9 is provided between the ultrasonic element array substrate 8 and the acoustic lens 10. A silicone-based adhesive used for the acoustic matching layer 9, the adhesive is cured so that the ultrasonic element array substrate 8 and the acoustic lens 10 are adhered to each other, and the cured adhesive functions as the acoustic matching layer. As mentioned above, the cured adhesive fills between the ultrasonic element array substrate 8 and the acoustic lens 10 without a gap.

The acoustic lens 10 is made of a resin such as a silicone resin. Acoustic impedance may be adjusted by adding silica to the silicone resin so as to change the specific gravity. The acoustic lens 10 is formed to have bending rigidity lower than that of the ultrasonic element array substrate 8.

The acoustic lens 10 efficiently guides ultrasonic waves which are transmitted from the ultrasonic elements of the ultrasonic element array substrate 8, and efficiently guides echo waves reflected and returned from a subject to the ultrasonic elements. The acoustic lens 10 protrudes in the −Z direction with a predetermined curvature. Ultrasonic waves output from the ultrasonic elements are collected at an examined location by the acoustic lens 10. The acoustic matching layer 9 alleviates mismatch of acoustic impedance between the ultrasonic elements and the acoustic lens 10. In other words, the acoustic matching layer 9 adjusts the acoustic impedance to be intermediate between the ultrasonic element array substrate 8 and the acoustic lens 10.

The fixation base 5 is made of a metal or a resin such as an acrylic resin or an ABS resin. The fixation base 5 has an area wider than that of the ultrasonic element array substrate 8, and has bending rigidity higher than that of the ultrasonic element array substrate 8. The bending rigidity of the acoustic lens 10 is lower than that of the ultrasonic element array substrate 8. As mentioned above, since the ultrasonic wave detection portion 4 has a structure of being fixed to the fixation base 5 having the bending rigidity higher than that of the ultrasonic element array substrate 8, the ultrasonic element array substrate 8 is reinforced, and thus it is possible to reduce concern that the ultrasonic element array substrate 8 may be damaged due to external force. Since the acoustic matching layer 9 is provided between the ultrasonic element array substrate 8 and the acoustic lens 10, there is an effect in which the acoustic lens 10 having the low bending rigidity and the acoustic matching layer 9 absorb external force, and thus the external force applied to the ultrasonic element array substrate 8 is reduced.

The head section 2 includes a casing 11, and the fixation base 5 is built into the casing 11. The fixation base 5 is connected to the casing 11 via a connector 12. A vibration control member 13 fills between the fixation base 5 and the casing 11. The connector 12 is made of an elastic material, and absorbs impact which is applied to the casing 11. The stress of the impact is hardly forwarded to the fixation base 5. The vibration control member 13 is a member having elastic modulus smaller than that of the connector 12. The vibration control member 13 has a function of attenuating vibration of the fixation base 5 when the fixation base 5 vibrates relative to the casing 11.

A material of the connector 12 is not particularly limited as long as the material has a spring property. A spring steel, a stainless steel, a copper alloy, a nickel alloy, a titanium alloy, and the like may be used. In the present embodiment, for example, the stainless steel is used as a material of the connector 12. A surface of the connector 12 may be plated so that anticorrosion is improved. The connector 12 is formed by processing a plate-shaped material by using a press device. A material of the vibration control member 13 is not particularly limited as long as the material has a function of alleviating vibration. Natural rubber, synthetic rubber, silicone rubber, gel-like members, and the like may be used. In the present embodiment, for example, the silicone rubber is used as a material of the vibration control member 13.

The casing 11 has an opening 11a on its surface on the −Z direction side. The ultrasonic wave detection portion 4 is exposed from the opening 11a. Thus, the ultrasonic wave detection portion 4 easily outputs ultrasonic waves outward of the casing 11. The holding section 3 is fixed to the casing 11 on the +Z direction side of the casing 11. The operator holds the holding section 3 and can easily change a direction of the ultrasonic wave detection portion 4. A wiring 14 is provided on the +Z direction side of the holding section 3. The ultrasonic probe 1 outputs a data signal of a reflected wave detected by the ultrasonic wave detection portion 4 to an external apparatus via the wiring 14.

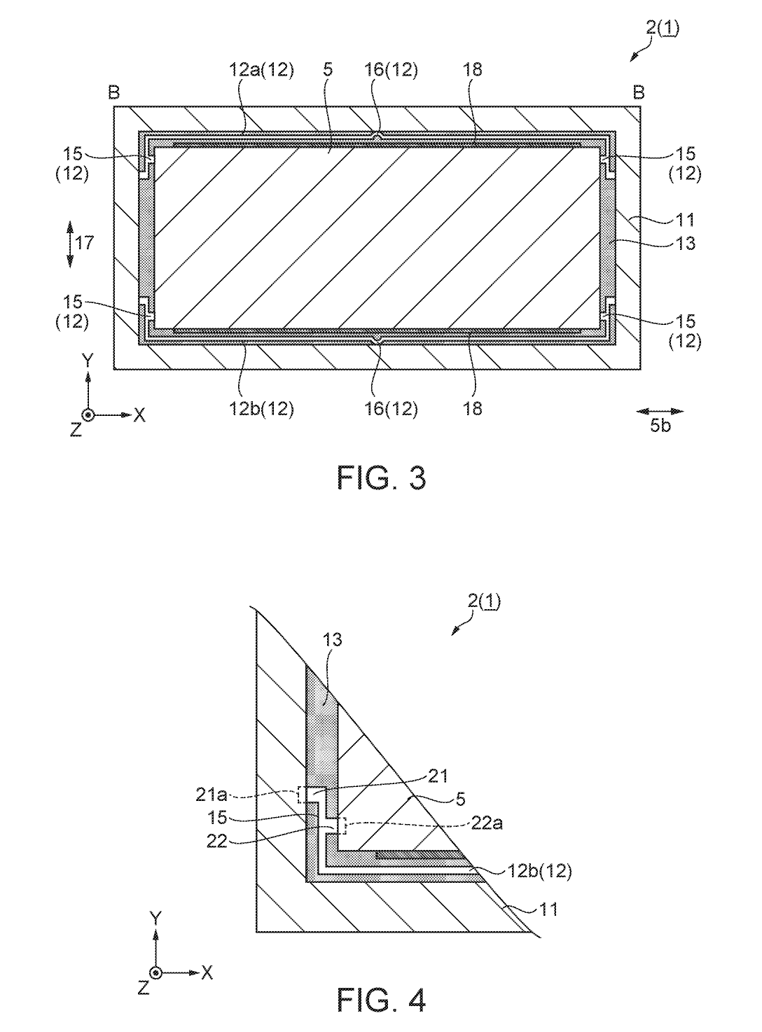

FIG. 3 is a schematic plan sectional view illustrating a structure of the head section, and is a view which is viewed from a surface side along the line B-B in FIG. 1. As illustrated in FIG. 3, the connector 12 is provided between the fixation base 5 and the casing 11. The connector 12 is formed of two portions such as an upper connector 12a and a lower connector 12b. The upper connector 12a is located on the +Y direction side of the fixation base 5, and the lower connector 12b is located on the −Y direction side of the fixation base 5.

Each of the upper connector 12a and the lower connector 12b includes first connection portions 15 and second connection portions 16. The connector 12 is provided with four first connection portions 15. The two first connection portions 15 are provided on the −X direction side of the fixation base 5, and the two first connection portions 15 are provided on the +X direction side of the fixation base 5. Consequently, even in a case where force acts between the fixation base 5 and the casing 11, relative swinging between the fixation base 5 and the casing 11 is reduced. In other words, the first connection portions 15 are connected to the casing 11 on both sides of the fixation base 5 in the third direction 5b. Two second connection portions 16 are provided. The one second connection portion 16 is provided on the −Y direction side of the fixation base 5, and the one second connection portion 16 is provided on the +Y direction side of the fixation base 5. Therefore, the fixation base 5 is connected to the casing 11 at six locations. The second connection portion 16 of the connector 12 is a portion from a corner in the −X direction to a corner in the +X direction in the fixation base 5. The second connection portions 16 are in contact with the center of the casing 11 in the X direction.

The first connection portion 15 is in contact with the fixation base 5 and the casing 11 and works so that relative positions between the fixation base 5 and the casing 11 do not vary. The second connection portions 16 are in contact with the casing 11. The second connection portion 16 of the upper connector 12a biases the casing 11 in the +Y direction side, and the second connection portion 16 of the lower connector 12b biases the casing 11 in the −Y direction side. When the directions in which the second connection portions 16 bias the casing 11 are referred to as a fourth direction 17, the connector 12 biases the casing 11 so as to define a position of the fixation base 5 in the fourth direction 17.

A flexible wiring 18 is provided on a surface of the fixation base 5 on the +Y direction side. The flexible wiring is located between the fixation base 5 and the upper connector 12a, and is separated from the upper connector 12a. A flexible wiring 18 is also provided on a surface of the fixation base 5 on the −Y direction side. The flexible wiring 18 is located between the fixation base 5 and the lower connector 12b, and is separated from the lower connector 12b. The flexible wirings 18 are connected to the ultrasonic element array substrate 8 and the wiring 14, and relay a data signal of a reflected wave output from the ultrasonic element array substrate 8.

FIG. 4 is a main portion schematic diagram illustrating a structure of the first connection portion, and is a view in which the first connection portion 15 is viewed from the +Z direction side. Since the four first connection portions 15 have the same shape and similarly work, only the first connection portion 15 on the −X direction side and −Y direct ion side will be described, and description of the other first connection portions 15 will be omitted. As illustrated in FIG. 4, the first connection portion 15 has a first beam 21 and a second beam 22. The first beam 21 extends in the −X direction side, and an end thereof is fixed to the casing 11. This location is referred to as a first fixation portion 21a. The second beam 22 extends in the +X direction side, and an end thereof is fixed to the fixation base 5. This location is referred to as a second fixation portion 22a.

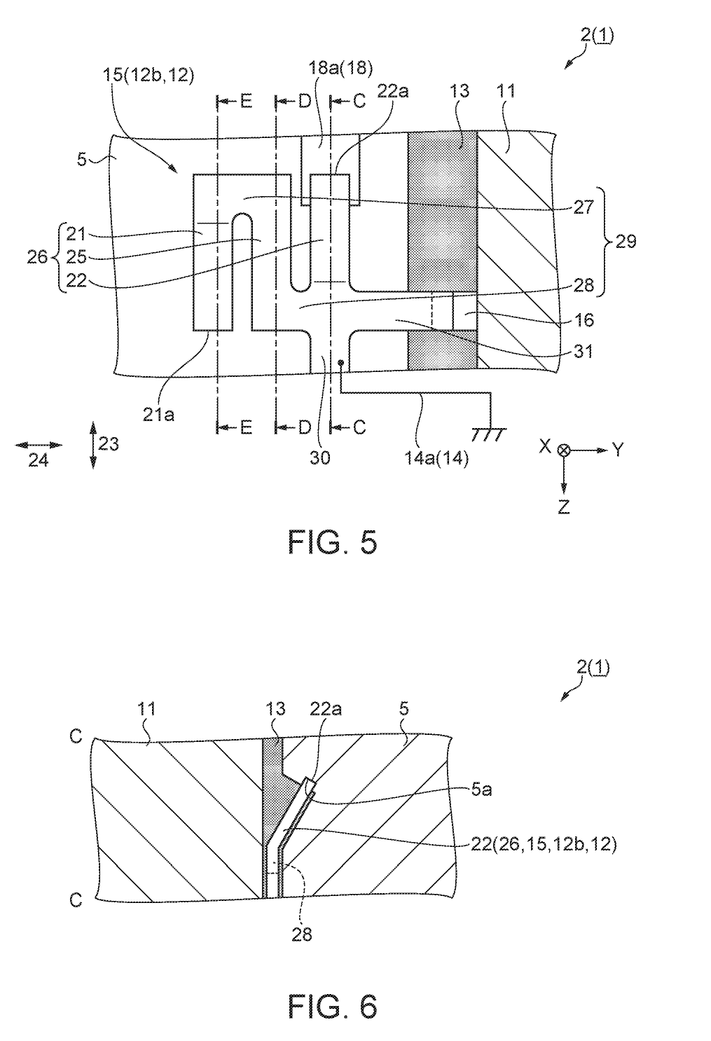

FIG. 5 is a main portion schematic diagram illustrating a structure of the first connection portion, and is a view in which the first connection portion 15 illustrated in FIG. 4 is viewed from the −X direction side. In FIG. 5, the Z direction is referred to as a first direction 23, and the Y direction is referred to as a second direction 24. The first beam 21 and the second beam 22 extend in the first direction 23. The first connection portion 15 has a third beam 25 extending in the first direction 23. The first beam 21, the second beam 22, and the third beam 25 form a first arm 26 extending in the first direction 23. The first arm 26 is a portion which is bent in the X direction and the Y direction.

A fourth beam 27 is provided on the −Z direction side of the first beam 21 and the third beam 25, and the fourth beam 27 connects the first beam 21 to the third beam 25. A fifth beam 28 is provided on the +Z direction side of the second beam 22 and the third beam 25, and the fifth beam 28 connects the second beam 22 to the third beam 25. The fourth beam 27 and the fifth beam 28 form a second arm 29 extending in the second direction 24. The second arm 29 is a portion which is bent in the X direction and the Z direction. The first direction 23 is a direction in which the first arm 26 extends, and the second direction 24 is a direction in which the second arm 29 extends. The first direction 23 and the second direction 24 are orthogonal to each other, but may obliquely intersect each other.

The first connection portion 15 is provided with the first arm 26 extending in the first direction 23, and the second arm 29 extending in the second direction 24. Consequently, the first connection portion 15 can displace relative positions between the first fixation portion 21a and the second fixation portion 22a respective three-dimensional directions.

The flexible wiring 18 is provided between the second fixation portion 22a and the fixation base 5. The first connection portion 15 is made of a conductive material. A ground wiring 18a provided in the flexible wiring 18 is electrically connected to the second fixation portion 22a. A sixth beam 30 which is connected to the second beam 22 and extends in the +Z direction is provided on the +Z direction side of the second beam 22. The sixth beam 30 is electrically connected to a ground wiring 14a included in the wiring 14. The first connection portion 15 relays the ground wiring 14a of the ultrasonic wave detection portion 4. Therefore, even when electrical noise is applied to the ultrasonic wave detection portion 4, the electrical noise is made to pass through the first connection portion 15 so as to be removed. A seventh beam 31 is connected to the fifth beam 28 on the +Y direction side, and the seventh beam 31 extends to the second connection portion 16. Since the first connection portion 15 is conductive and has a sectional area larger than that of the wiring, the first connection portion 15 has low resistance and can reduce influence of electrical noise.

FIG. 6 is a main portion schematic diagram for explaining a structure of the second fixation portion of a second beam. As illustrated in FIG. 6, a hole 5a is formed in the fixation base 5, and the second fixation portion 22a is inserted into the hole 5a. There is a gap between the second beam 22 and the fixation base 5 in regions other than the second fixation portion 22a inserted into the hole 5a, and thus the second beam 22 can be bent. In other words, the second fixation portion 22a is in contact with the fixation base 5, and movement of the connector 12 is restricted.

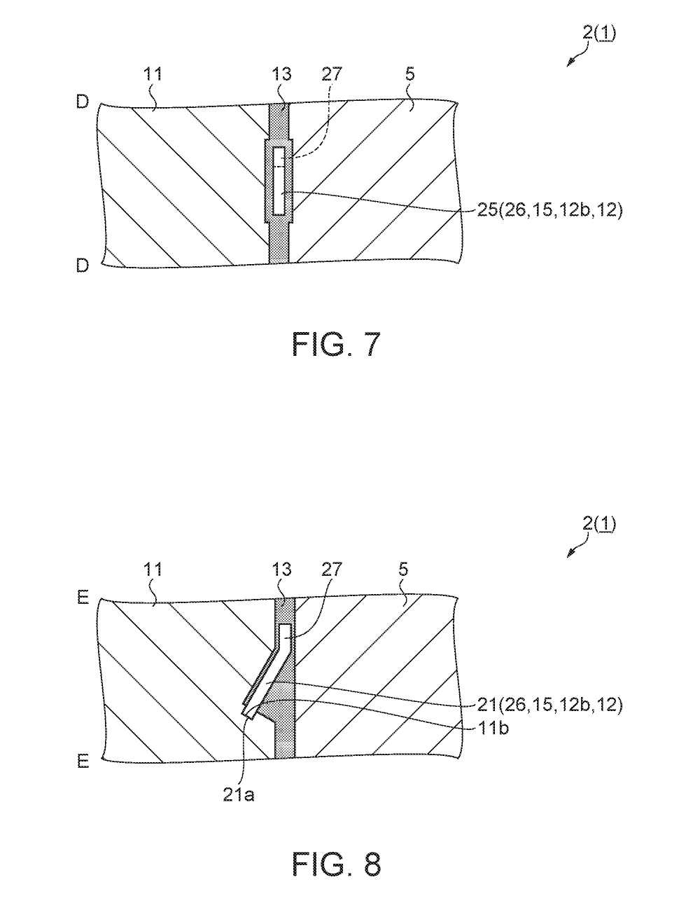

FIG. 7 is a main portion schematic diagram for explaining a structure of the third beam. As illustrated in FIG. 7, the third beam 25 has a gap with the fixation base 5, and also has a gap with the casing 11. Thus, the third beam 25 can be bent.

FIG. 8 is a main portion schematic diagram for explaining a structure of the first fixation portion of the first beam. As illustrated in FIG. 8, a hole 11b is formed in the casing 11, and the first fixation portion 21a is inserted into the hole 11b. There is a gap between the first beam 21 and the casing 11 in regions other than the first fixation portion 21a inserted into the hole 11b, and thus the first beam 21 can be bent. In other words, the first fixation portion 21a is in contact with the casing 11, and movement of the connector 12 is restricted.

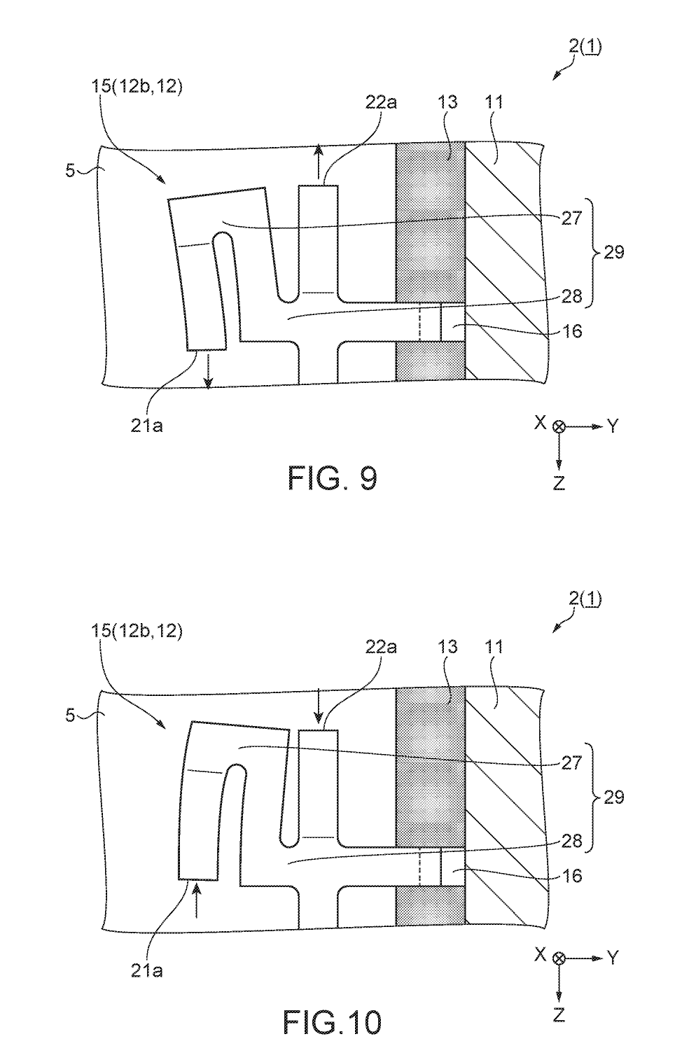

Next, an operation of the first connection portion 15 will be described. FIGS. 9 to 14 are schematic diagrams for explaining an operation of the first connection portion. As illustrated in FIG. 9, when an external force is applied to the casing 11 so that the fixation base 5 is displaced in the −Z direction, the second arm 29 is deformed, and thus a distance between the first fixation portion 21a and the second fixation portion 22a in the Z direction is increased. At this time, the second arm 29 has the spring property, and thus absorbs the external force so as to reduce the displacement. Similarly, as illustrated in FIG. 10, when an external force is applied to the casing 11 so that the fixation base 5 is displaced in the +Z direction, the second arm 29 is deformed, and thus a distance between the first fixation portion 21a and the second fixation portion 22a in the Z direction is decreased. Also at this time, the second arm 29 has the spring property, and thus absorbs the external force so as to reduce the displacement.

The ultrasonic wave detection portion 4 is exposed from the casing 11. The ultrasonic wave detection portion 4 is pressed onto a subject. When the ultrasonic wave detection portion 4 is pressed, force is forwarded to the fixation base 5. An external force is applied to the casing 11 so that the fixation base 5 is displaced in the +Z direction. At this time, the first connection portion 15 receives the force. Therefore, the ultrasonic wave detection portion 4 can be prevented from being moved to the inside of the casing 11 when the ultrasonic wave detection portion 4 is pressed onto the subject.

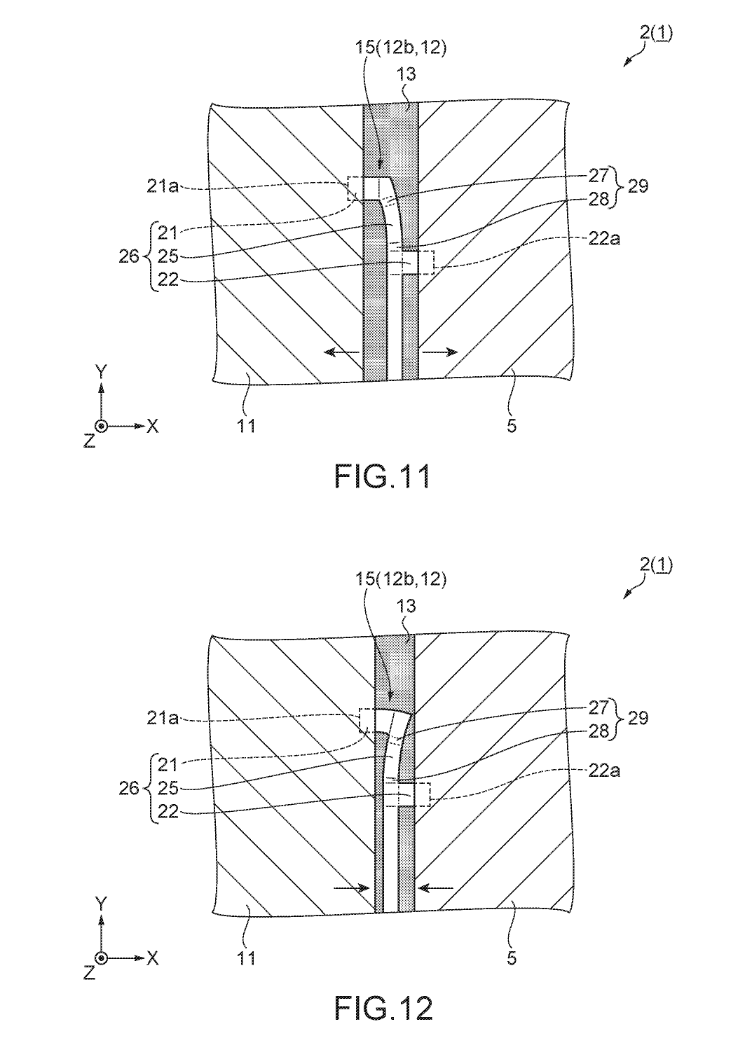

As illustrated in FIG. 11, when an external force is applied to the casing 11 so that the fixation base 5 is displaced in the X direction, the first arm 26 and the second arm 29 are deformed, and thus a distance between the first fixation portion 21a and the second fixation portion 22a in the X direction is increased. At this time, the first arm 26 and the second arm 29 have the spring property, and thus absorb the external force so as to reduce the displacement. Similarly, as illustrated in FIG. 12, when an external force is applied to the casing 11 so that the fixation base 5 is displaced in the −X direction, and the first arm 26 and the second arm 29 are deformed, and thus a distance between the first fixation portion 21a and the second fixation portion 22a in the X direction is decreased. Also at this time, the first arm 26 and the second arm 29 have the spring property, and thus absorb the external force so as to reduce the displacement.

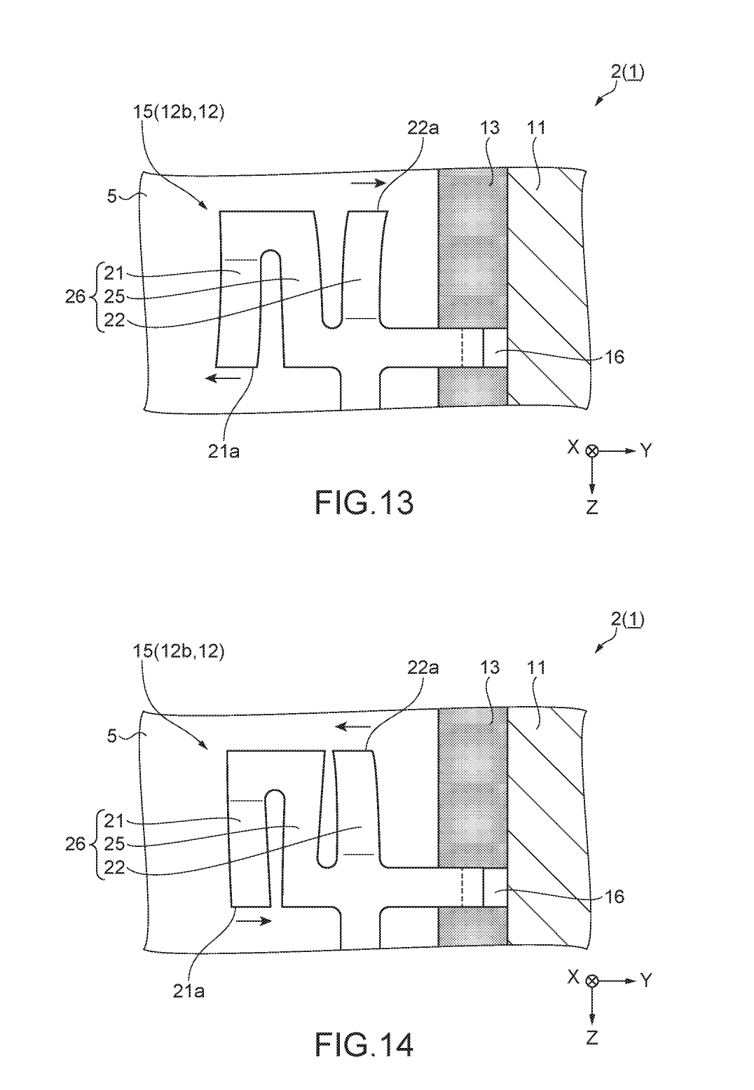

As illustrated in FIG. 13, when an external force is applied to the casing 11 so that the fixation base 5 is displaced in the +Y direction, the first arm 26 is deformed, and thus a distance between the first fixation portion 21a and the second fixation portion 22a in the Y direction is increased. At this time, the first arm 26 has the spring property, and thus absorbs the external force so as to reduce the displacement. Similarly, as illustrated in FIG. 14, when an external force is applied to the casing 11 so that the fixation base 5 is displaced in the −Y direction, the first arm 26 is deformed, and thus a distance between the first fixation portion 21a and the second fixation portion 22a in the Y direction is decreased. Also at this time, the first arm 26 has the spring property, and thus absorbs the external force so as to reduce the displacement.

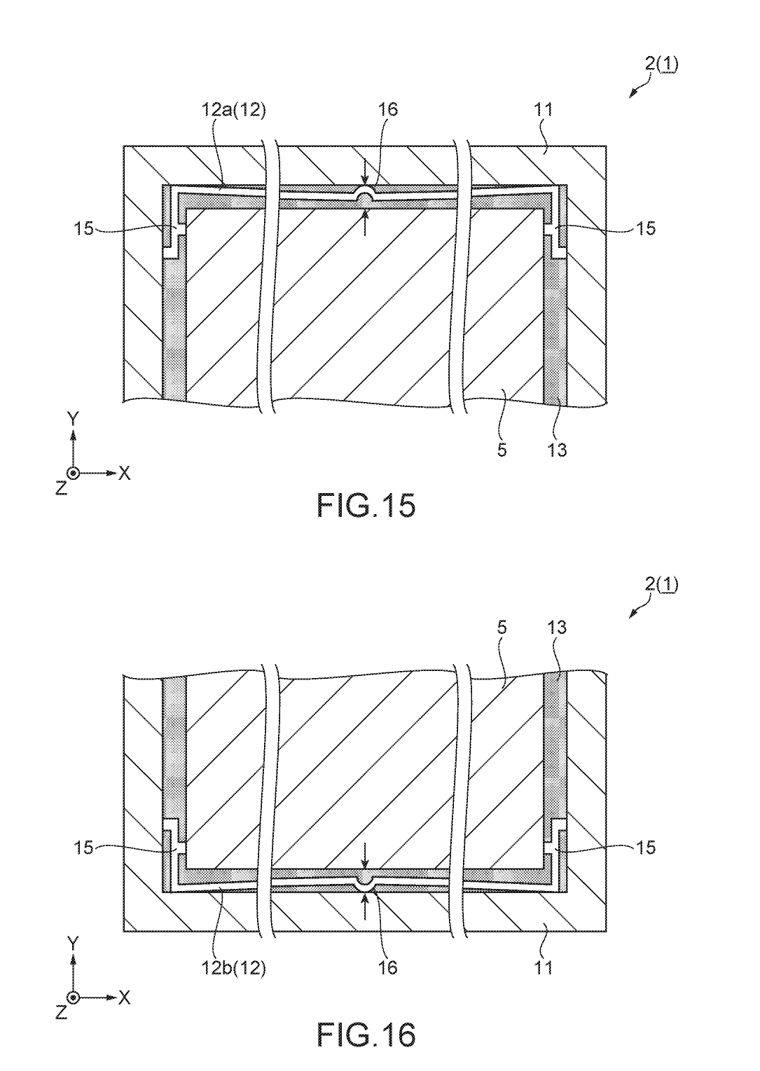

Next, an operation of the second connection portion 16 will be described. FIGS. 15 and 16 are schematic diagrams for explaining an operation of the second connection portion. As illustrated in FIG. 15, when an external force is applied to the casing 11 so that the fixation base 5 is displaced in the +Y direction, the upper connector 12a is deformed, and thus the second connection portion 16 presses the casing 11. At this time, the upper connector 12a has the spring property, and thus absorbs the external force so as to reduce the displacement. Similarly, as illustrated in FIG. 16, when an external force is applied to the casing 11 so that the fixation base 5 is displaced in the −Y direction, the lower connector 12b is deformed, and thus the second connection portion 16 presses the casing 11. At this time, the lower connector 12b has the spring property, and thus absorbs the external force so as to reduce the displacement.

The vibration control member 13 attenuating vibration is provided between the fixation base 5 and the casing 11. The vibration control member 13 attenuates vibration. Therefore, the ultrasonic probe 1 can reduce influence of vibration caused by impact when the impact is applied thereto. The vibration control member 13 is provided on the surface of the fixation base 5 in the X direction and the Y direction. It is also possible to attenuate vibration caused by a shearing force when impact is applied in the Z direction. Therefore, it is possible to attenuate vibration in any direction.

As described above, according to the present embodiment, the following effects can be achieved.

(1) According to the present embodiment, the ultrasonic probe 1 includes the ultrasonic wave detection portion 4, the fixation base 5, the casing 11, and the first connection portion 15. The ultrasonic wave detection portion 4 transmits an ultrasonic wave and receives a reflected wave. The ultrasonic wave detection portion 4 is fixed to the fixation base 5. The ultrasonic wave detection portion 4 is exposed from the casing 11, and the fixation base 5 is built into the casing 11. The first connection portion 15 is elastic, and connects the fixation base 5 to the casing 11.

The first connection portion 15 includes the first arm 26 extending in the first direction 23, and the second arm 29 extending in the second direction 24 orthogonal to the first direction 23. The first arm 26 and the second arm 29 are elastic and thus function as springs. The second fixation portion 22a which is one end of the first arm 26 is in contact with the fixation base 5, and the first fixation portion 21a which is one end of the second arm 29 is in contact with the casing 11. Therefore, the first connection portion 15 is deformed when impact is applied to the casing 11, and thus it is possible to reduce impact which is applied to the ultrasonic wave detection portion 4 from the casing 11.

The first arm 26 and the second arm 29 are located between the fixation base 5 and the casing 11, and thus the first direction 23 and the second direction 24 are the same as directions in which a surface of the fixation base 5 and a surface of the casing 11 facing each other extend. Therefore, a gap between the fixation base 5 and the casing 11 can be reduced, and thus the ultrasonic probe 1 can be reduced in size.

(2) According to the present embodiment, the fixation base 5 has the shape which is long in the third direction 5b. The first connection portion 15 is connected to the casing 11 on both sides of the fixation base 5 in the third direction 5b. Consequently, the first connection portion 15 can favorably reduce impact in the third direction 5b. The ultrasonic probe 1 includes the second connection portion 16. The second connection portion 16 connects the fixation base 5 to the casing 11 in the fourth direction 17 orthogonal to the third direction 5b, and is elastic. Consequently, the second connection portion 16 can favorably reduce impact in the fourth direction 17. As a result, the ultrasonic probe 1 can reduce impact in two directions including the third direction 5b and the fourth direction 17.

(3) According to the present embodiment, the vibration control member 13 is provided between the fixation base 5 and the casing 11, and the vibration control member 13 attenuates vibration. Therefore, when impact is applied to the ultrasonic probe 1, it is possible to reduce influence of vibration caused by the impact.

(4) According to the present embodiment, the four first connection portions 15 are provided. The fixation base 5 is held on the casing 11 at four locations via the first connection portions 15, and thus it is possible to reduce swinging of the fixation base 5 due to impact.

(5) According to the present embodiment, the ultrasonic wave detection portion 4 is exposed from the casing 11. The ultrasonic wave detection portion 4 is pressed onto a subject. Force applied when the ultrasonic wave detection portion 4 is pressed is received by the first connection portion 15. Therefore, the ultrasonic probe 1 can prevent the ultrasonic wave detection portion 4 from being moved inside the casing 11 when the ultrasonic wave detection portion 4 is pressed onto the subject.

(6) According to the present embodiment, the first connection portion 15 is conductive. The first connection portion 15 relays the ground wiring 18a and the ground wiring 14a of the ultrasonic wave detection portion 4. Therefore, even when electrical noise is applied to the ultrasonic wave detection portion 4, the electrical noise is made to pass through the first connection portion 15 so as to be removed.

### Second Embodiment

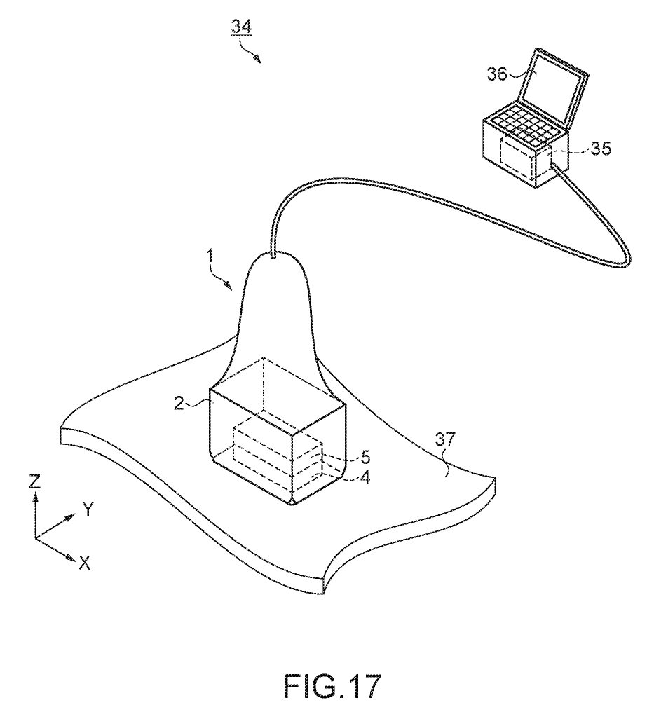

Next, an embodiment of an ultrasonic image apparatus using the ultrasonic probe 1 will be described with reference to FIG. 17. FIG. 17 is a schematic perspective view illustrating a structure of the ultrasonic image apparatus. Description of the same constituent elements as in the first embodiment will be omitted.

In other words, in the present embodiment, as illustrated in FIG. 17, an ultrasonic image apparatus 34 includes the ultrasonic probe 1, an image data calculation unit 35, and an image display unit 36. The ultrasonic probe 1 transmits ultrasonic waves to a subject 37. The ultrasonic probe 1 receives reflected waves of the ultrasonic waves which are reflected inside the subject 37, and outputs a reflected wave signal to the image data calculation unit 35. The image data calculation unit 35 calculates tomographic image data of the subject 37 by using the reflected wave signal. The image data calculation unit 35 outputs a tomographic image to the image display unit 36. The image display unit 36 displays the tomographic image of the subject 37.

As the ultrasonic probe 1, the ultrasonic probe 1 according to the first embodiment is used. The ultrasonic probe 1 has impact resistance, a small size, and good operability. Therefore, the ultrasonic image apparatus 34 can be provided as an apparatus including the ultrasonic probe 1 having impact resistance, a small size, and good operability.

The present embodiment is not limited to the above-described embodiment, and may be variously changed or altered by a person skilled in the art within the technical spirit of the invention. Hereinafter, modification examples will be described.

### Modification Example 1

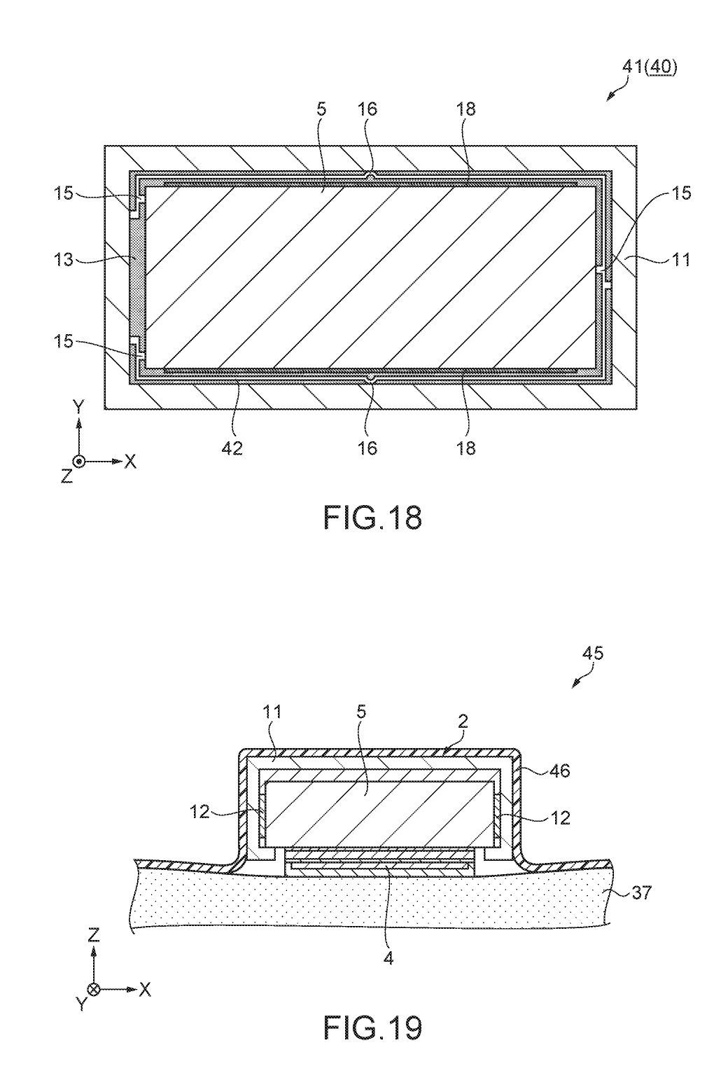

The four first connection portions 15 are provided in the connector 12 in the first embodiment. FIG. 18 is a schematic plan sectional view for explaining arrangement of a first connection portion. As illustrated in FIG. 18, in a head section 41 provided in an ultrasonic probe 40, a connector 42 is provided between a fixation base 5 and a casing 11.

The connector 42 includes two first connection portions 15 which are provided on the −X direction side, and a single first connection portion 15 which is provided on the +X direction side. As mentioned above, the first connection portions 15 may be the first connection portions 15 provided at three locations. Also in this case, it is possible to reduce that the fixation base 5 is swung with respect to the casing 11 when impact is applied to the ultrasonic probe 40. In a case where the number of first connection portions 15 is five or larger, it is possible to further reduce that the fixation base 5 is swung with respect to the casing 11.

### Modification Example 2

In the first embodiment, the holding section 3 is provided in the head section 2 of the ultrasonic probe 1. FIG. 19 is a schematic side sectional view for explaining a structure of an ultrasonic probe. As illustrated in FIG. 19, the head section 2 without the holding section 3 may be an ultrasonic probe 45. In this case, the head section 2 is fixed to the subject 37 by using a sticky tape 46. A state of an internal change of the subject 37 over time can be observed.

Also in a case of the ultrasonic probe 45, impact may be applied to the ultrasonic probe 45 due to falling. In this case, impact applied to the casing 11 can be reduced by the connector 12. Therefore, it is possible to provide the ultrasonic probe 45 having impact resistance, a small size, and good operability.

### Modification Example 3

In the first embodiment, the connector 12 is made of a plate-shaped material. The connector 12 may be made of a linear material by using a wire forming device. In a case where the number of products to be manufactured is small, it is possible to reduce cost for a blanking mold by using a linear material instead of a plate material.

### Modification Example 4

In the first embodiment, the first arm 26 is formed of three portions including the first beam 21, the second beam 22, and the third beam 25 extending in the first direction 23. The first arm 26 may be formed of one or two beams, and may be formed of four or more beams. The second arm 29 is formed of the fourth beam 27 and the fifth beam 28 extending in the second direction 24. The second arm 29 may be formed of a single beam, and may be formed of three or more beams. The arms may be configured to be easily designed. The first arm 26 and the second arm 29 may have curved shapes. The arms may include portions extending in two different directions.

### Modification Example 5

In the first embodiment, as illustrated in FIG. 6, the second fixation portion 22a is fixed to the fixation base 5. A groove extending in the Y direction may be provided in the fixation base 5, and the second fixation portion 22a may be in contact with the groove. Force applied in the −Z direction from the fixation base 5 may be received by the second fixation portion 22a. As illustrated in FIG. 8, the first fixation portion 21a is fixed to the casing 11. A groove extending in the Y direction may be provided in the casing 11, and the first fixation portion 21a may be in contact with the groove. Force applied in the +Z direction from the first beam 21 may be received by the casing 11. With this structure, the fixation base 5, the connector 12, and the casing 11 can be easily assembled.

Also in this structure, impact can be absorbed in both of positive and negative directions in each of the X direction and the Y direction by using the spring of the connector 12. Regarding the Z direction, it is possible to reduce impact applied to the fixation base 5 and a load in use in the −Z direction. Regarding the Z direction, impact applied to the fixation base 5 in the +Z direction cannot be reduced, and thus an elastic member is preferably provided between the fixation base 5 and the casing 11. Therefore, also in this modification example, the first connection portion 15 is deformed when impact is applied to the casing 11, and thus it is possible to reduce impact applied to the ultrasonic wave detection portion 4 from the casing 11.

Preferably, the second fixation portion 22a of the second beam 22 is in contact with the fixation base 5, and the fifth beam 28 side is separated from the fixation base 5. Consequently, the second beam 22 can act as a beam having the spring property. Similarly, preferably, the first fixation portion 21a of the first beam 21 is in contact with the casing 11, and the fourth beam 27 side is separated from the casing 11. Consequently, the first beam 21 can act as a beam having the spring property.

The entire disclosure of Japanese Patent Application No. 2015-229388, filed on Nov. 25, 2015 is expressly incorporated by reference herein.

What is claimed is:

1. An ultrasonic probe comprising:
an ultrasonic wave detection portion that includes an ultrasonic element performing at least one of transmission and reception of an ultrasonic wave;
a fixation base to which the ultrasonic wave detection portion is fixed;
a casing from which the ultrasonic wave detection portion is exposed and into which the fixation base is built; and
a first connection portion that connects the fixation base to the casing, and is elastic,
wherein the first connection portion includes a first arm extending in a first direction and a second arm extending in a second direction intersecting the first direction,
wherein one end of the first connection portion is in contact with the fixation base, and the other end of the first connection portion is in contact with the casing, and
wherein the first direction and the second direction are the same as directions in which a surface of the fixation base and a surface of the casing facing each other extend.

2. The ultrasonic probe according to claim 1,
wherein the fixation base is long in a third direction,
wherein the first connection portion is connected to the casing on both sides of the fixation base in the third direction, and
wherein the ultrasonic probe further includes a second connection portion that connects the fixation base to the casing in a fourth direction intersecting the third direction, and that is elastic.

3. The ultrasonic probe according to claim 1, further comprising:
a vibration control member that is provided between the fixation base and the casing so as to attenuate vibration.

4. The ultrasonic probe according to claim 1,
wherein the first connection portion is provided at three or more locations.

5. The ultrasonic probe according to claim 1,
wherein the ultrasonic wave detection portion is exposed from the casing, and force applied when the ultrasonic wave detection portion is pressed is received by the first connection portion.

6. The ultrasonic probe according to claim 1,
wherein the first connection portion is conductive, and relays a ground wiring of the ultrasonic wave detection portion.

7. An ultrasonic image apparatus comprising:
the ultrasonic probe according to claim 1;
an image data calculation unit that calculates tomographic image data of a subject by using a reflected wave signal output from the ultrasonic probe; and
an image display unit that displays a tomographic image of the subject on the basis of a result calculated by the image data calculation unit.

8. An ultrasonic image apparatus comprising:
the ultrasonic probe according to claim 2;
an image data calculation unit that calculates tomographic image data of a subject by using a reflected wave signal output from the ultrasonic probe; and
an image display unit that displays a tomographic image of the subject on the basis of a result calculated by the image data calculation unit.

9. An ultrasonic image apparatus comprising:
the ultrasonic probe according to claim 3;
an image data calculation unit that calculates tomographic image data of a subject by using a reflected wave signal output from the ultrasonic probe; and
an image display unit that displays a tomographic image of the subject on the basis of a result calculated by the image data calculation unit.

10. An ultrasonic image apparatus comprising:
the ultrasonic probe according to claim 4;
an image data calculation unit that calculates tomographic image data of a subject by using a reflected wave signal output from the ultrasonic probe; and
an image display unit that displays a tomographic image of the subject on the basis of a result calculated by the image data calculation unit.

11. An ultrasonic image apparatus comprising:
the ultrasonic probe according to claim 5;
an image data calculation unit that calculates tomographic image data of a subject by using a reflected wave signal output from the ultrasonic probe; and
an image display unit that displays a tomographic image of the subject on the basis of a result calculated by the image data calculation unit.

12. An ultrasonic image apparatus comprising:
the ultrasonic probe according to claim 6;
an image data calculation unit that calculates tomographic image data of a subject by using a reflected wave signal output from the ultrasonic probe; and
an image display unit that displays a tomographic image of the subject on the basis of a result calculated by the image data calculation unit.
